(19) United States
(12) Patent Application Publication
(10) Pub. No.: US 2014/0330116 A1
(43) Pub. Date: Nov. 6, 2014

(54) SYSTEMS AND METHODS FOR SIMULTANEOUS MULTI-DIRECTIONAL IMAGING FOR CAPTURING TOMOGRAPHIC DATA

(71) Applicant: The Trustees of Columbia University in the Ciyt of New York, New York, NY (US)

(72) Inventors: Andreas H. Hielscher, Brooklyn, NY (US); Jong Hwan Lee, Seoul (KR)

(21) Appl. No.: 14/356,932

(22) PCT Filed: Nov. 8, 2012
(86) PCT No.: PCT/US2012/064245

§ 371 Date: May 8, 2014

Related U.S. Application Data
(60) Provisional application No. 61/557,045, filed on Nov. 8, 2011.

(51) Int. Cl. A61B 5/00

(52) U.S. Cl. CPC A61B 5/0073; A61B 5/0077
USPC 600/425

(57) ABSTRACT

Devices, systems, and method for tomographic imaging are described in which light transmitted and backscattered surface light is imaged by an optical system that minimizes reflection back to the target object while providing the ability to direct surface image rays to a single imaging device. In an embodiment, a curved reflector surrounds, fully or partly, a target object but is angled to reflect light toward the imaging camera.

### STATEMENT REGARDING FEDERALLY SPONSORED RESEARCH

This invention was made with government support under NCI-4R33CA118666 awarded by National Cancer Institute. The government has certain rights in the invention.

### BACKGROUND

Diffuse Optical Tomography (DOT) employs optical imaging of the surface of an interrogated object to determine the three-dimensional distribution of chromophores (or fluorophores if used) in the object. Objects may include laboratory animals and human body parts. The three-dimensional distribution may allow bio-markers and physiological parameters such as oxygen and certain molecules of interest to be followed. The technique is non-invasive and does not involve harmful radiation.

Optical tomography techniques such as diffuse optical tomography, fluorescence tomography and bioluminescence tomography are non-invasive techniques for in vivo diagnostic studies. Because these optical tomographic techniques can provide three-dimensional and quantitative information of biological factors in the living systems, they are regarded as important tools for biomedical research and medical diagnosis.

Optical tomographic instrumentation consists of apparatus for illuminating the interrogated object and equipment for measuring emitted photons from the surface of a subject. Illumination may provide epi-illumination or trans-illumination of the target object. A computer employs a numerical reconstruction algorithm to generate three-dimensional information from the measured data. Some DOT systems employ a CCD camera to capture high resolution representation of the surface-emitted photons. This is distinct from fiber-based systems that contact the surface and produces many more points of measurement.

Known devices have been used to capture images with both epi- and trans-illuminated target objects with processing to combine image data for both. To accomplish this, approaches such as rotating the target object with a fixed camera, using multiple cameras aimed in several different directions, and placing a pyramidal or conical mirror around the subject and aiming a camera to capture the reflected image therefrom. Especially, in the case of a conical mirror, the entire surface of a subject can be observed simultaneously and more emission photons can be detected compared with the flat minor scheme.

### SUMMARY

Objects and advantages of embodiments of the disclosed subject matter will become apparent from the following description when considered in conjunction with the accompanying drawings.

In embodiments, two consecutive mirror reflections are arranged to reduce backscattering from an illuminated object that further illuminates the object and interferes with the image data that is captured. A primary reflector is positioned remote from the object and at least partly surrounding it while a secondary reflector reflects an image from the primary reflector to a camera. The use of two reflectors in this embodiment facilitates the positioning and orienting of the primary reflector such that backscatter is reduced or eliminated.

### BRIEF DESCRIPTION OF THE DRAWINGS

Embodiments will hereinafter be described in detail below with reference to the accompanying drawings, wherein like reference numerals represent like elements. The accompanying drawings have not necessarily been drawn to scale. Where applicable, some features may not be illustrated to assist in the description of underlying features.

FIGS. 1A and 1B show a reflective cone type device used for capturing optical tomographic images according to the prior art.

FIG. 2 shows a first multiple reflection device for capturing tomographic imaging data according to an embodiment of the disclosed subject matter.

FIGS. 3A and 3B show a second multiple reflection device for capturing tomographic imaging data and adapted for use in capturing an image of breast tissue or similar body part according to an embodiment of the disclosed subject matter.

FIG. 4 shows a third multiple reflection device for capturing tomographic imaging data according to an embodiment of the disclosed subject matter.

FIG. 5A shows a fourth multiple reflection device for capturing tomographic data showing features for epi and trans illumination that may be used with other embodiments.

FIG. 5B shows a fourth multiple reflection device for capturing tomographic data showing features for supporting a scanned article that may be used with other embodiments.

FIGS. 6A and 6B illustrate principals of operation of an imaging device according to an embodiment of the disclosed subject matter.

FIGS. 7A and 7B illustrate a surface scanning and tomographic imaging system according to an embodiment of the disclosed subject matter.

### DETAILED DESCRIPTION

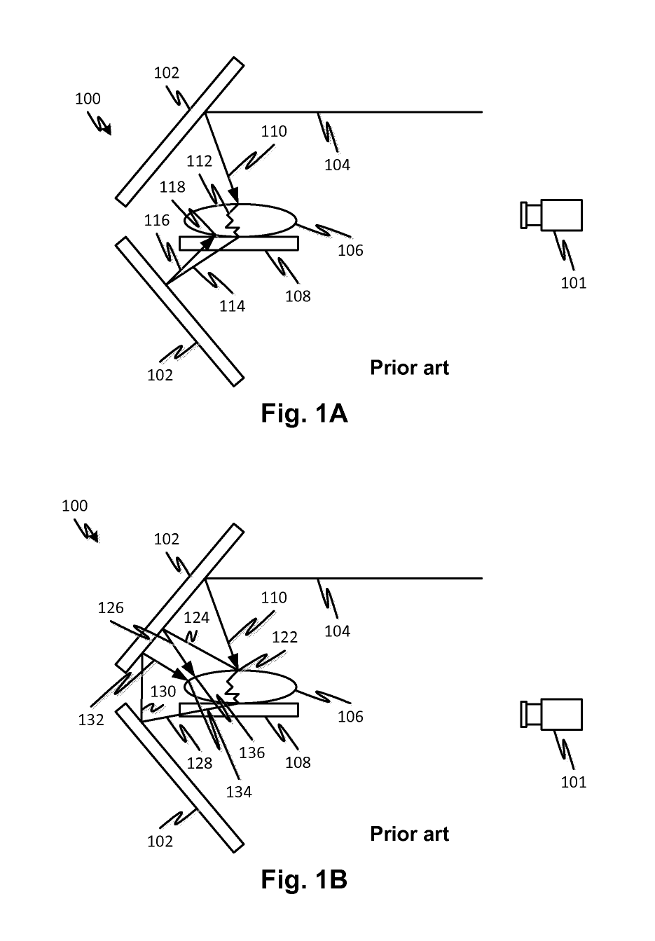

FIGS. 1A and 1B show a reflective cone type device 100 used for capturing optical tomographic images according to the prior art. A target object 106 is supported on a stage 108, which may be optically transparent. An illumination beam 104, such as a low power laser, is directed by reflection by the conical mirror 102 as indicated at 110 to many points on the surface of the object 106, with illumination of a single point being shown. Light penetrates the object 106 and scatters within as indicated at 112. Transmitted light 114 may reflect 116 from the conical mirror 102 and illuminate the target object as indicated at 118. Of course this happens at many points creating a diffuse illumination of the object 106 by backscattered light. As shown in FIG. 1B, the primary illumination beam may also produce direct reflections 124 which are backscattered either directly 126 by the mirror 102 or indirectly 130, 132 to illuminate the object 106 at various points such as 134, 136. Back illumination by transmitted light as illustrated in FIG. 1A is generally not as serious a concern since the illuminated light scatters and is attenuated by absorption as it passes through the target body. As in FIG. 1B, back illumination by reflected light is typically stronger. Because the intensity is strong, and its first, second, or third (etc.) reflections back and forth between the target and the mirror and/or a target can significantly affect the measured data.

As illustrated in FIG. 1A and 1B, because a target object 106 is placed inside of a highly reflective cone mirror and the source is illuminated on the surface of a subject by the conical mirror, the back reflections between the surface of a subject and a conical mirror, which has a negative effect on reconstruction, take place. This can reduce the accuracy and precision of the data measurement and the reconstruction results. The disclosed subject matter reduces this effect and provides the flexibility for combining the optical imaging system with other modalities. The present disclosure describes an imaging system for obtaining non-contact multi-directional optical tomographic data from a subject such as, but not limited to, small animals (e.g. mice or rats), hands, feet or body parts, for example, human breasts.

In embodiments, an imaging scheme uses two consecutive mirror reflections as illustrated in FIG. 2. The first mirrors 204 near a subject reflect the surface images from an object 202 into the second mirrors 206 which reflect the image to a CCD camera 210 so that all reflected images are obtained in a single picture. Thus, primary transmitted or reflected light from the object surface 202 indicated by beam 208 (it being understood that light emanates or is reflected in multiple directions at various points of the surface simultaneously) is reflected by the first mirror (or mirrors) 204 to a second mirror 206 resulting in light 212 being directed to the camera 210. The mirrors 204 and 206 may be circular cone segments, for example, or pyramidal or piecewise conical with or without discontinuities, or any kind of fully or partially circumnavigating structure that can reflect light from multiple angles.

A controller 214 may control an illumination source 209 such as a laser and cameras 201 and 210. The controller 214 may receive image data from the cameras 201 and 210. The camera 210 may be used to capture a fully image of the object 202 surface. The surface projection on CCD of the camera 210 will be distorted but in a predictable manner and the calculation of surface-emitted photon flux for each surface portion of the object 202 to a three-dimensional model (mesh) thereof in the controller 214 can be done with high accuracy since the geometry of the system is known. Camera 201 may be used to capture the precise surface geometry of the object 202 for modeling of the object 202. The light source 209 may be positioned at various locations depending on the shape of the object 202. The light source 209 may be supported on a rotating or movable gantry to illuminate portions of the object 202 that face in different directions. Alternatively, or in addition, multiple light sources 209 may be used.

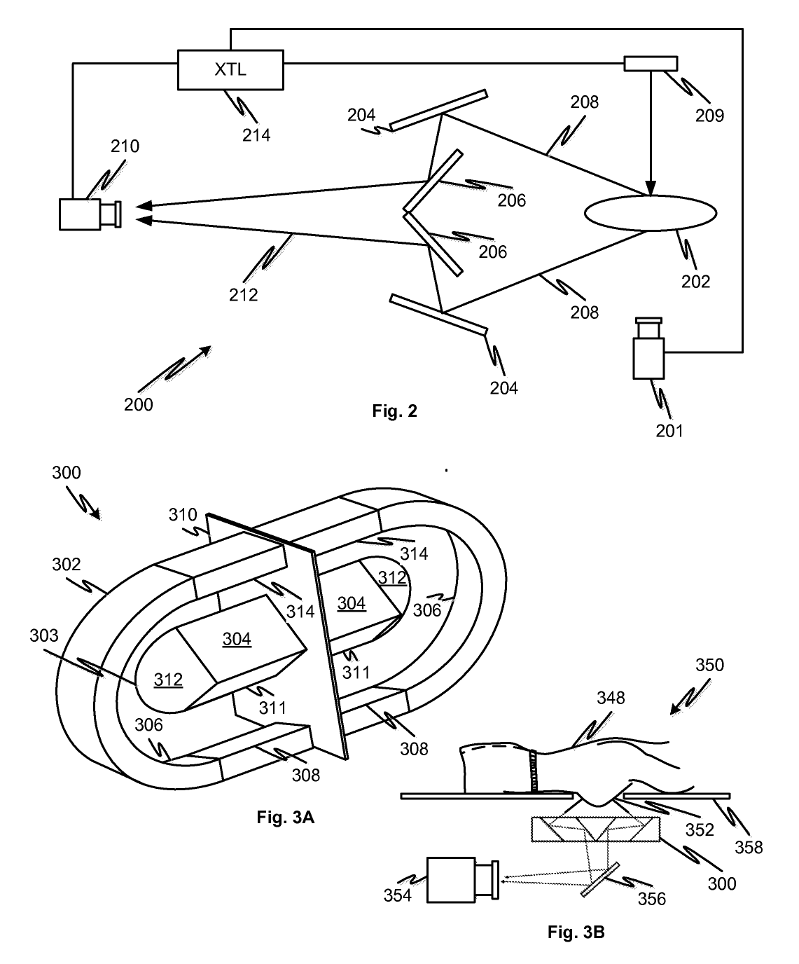

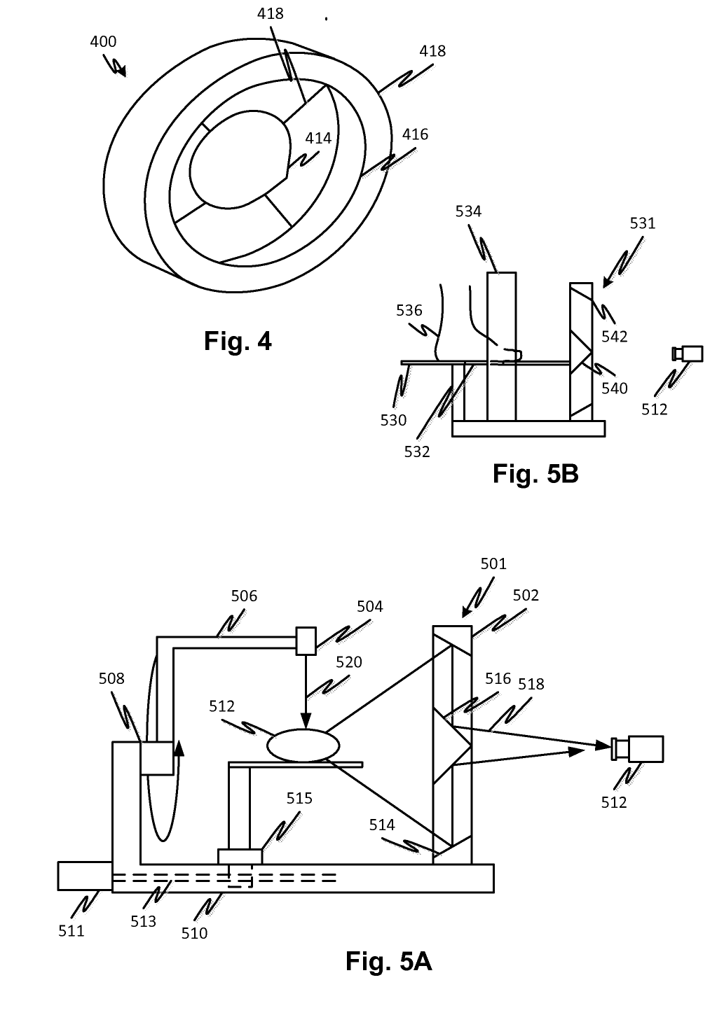

The geometry of the reflection scheme of FIG. 2 may be modified according to the shape of the target object 202. Depending on the surface geometry of the target, the shape of the first and the second mirror can be flat or conical or a combination of flat and conical shape. In embodiments, for example, a conical mirror pair is used as shown in FIG. 4 and discussed below. This may be suitable for small animal imaging. A flat minor scheme may be used for imaging relatively flat body parts such as a hand or foot as illustrated in FIG. 5B and discussed below. In addition, a combination of flat and conical mirrors may be used as illustrated in FIG. 3 and discussed below, for example, to image breast surfaces. In embodiments, the camera 201 may also serve other functions such as an additional surface photon emission data for the generation of tomographic data. The controller, multiple cameras, and illumination source features described with respect to FIG. 2 may be provided in the other embodiments described herein.

FIGS. 3A and 3B show an imaging device 300 with first and second reflector surfaces that include flat and curved surfaces. Flat minors 308 and 314 form portions of a primary reflector and conical portions 306 are contiguous with the flat portions 308. The secondary reflector is also made up of flat and curved portions. A flat portion 304 is contiguous with a conical portion 312. A planar support 310 may be light-absorbing to block the crosstalk of light between two breasts. The primary reflectors may be supported on a single rigid hollow structure 302 to provide rigidity and precise location of the reflecting surfaces supported thereon. Similarly for structure 303. Note that in alternative embodiments, the secondary reflector is located outside the space surrounded by the primary reflector with appropriate orientation of the reflective surfaces, camera, and target object.

The imaging device 300 may be used to image one or two breasts simultaneously. The target breast may be positioned behind the apparatus 300 from the vantage of the view of the figure and facing the apparatus 300. This orientation is such that light from the target strikes reflectors 306, 314, and 308, first and then reflectors 304, 311, and 312 to be directed at the camera which is on the near side of the apparatus 300 with respect to the vantage of the viewer of the figure. In embodiments, the apparatus 300 may be positioned by a system 350 in a horizontal orientation below a bed 358 supporting a patient 348 with openings for the breasts 352 to hang through as shown in FIG. 3B. A reflector 356 may permit a camera 354 to be positioned horizontally.

In FIG. 4, apparatus 400 is a lower aspect ratio imaging device than that of FIG. 3. A single conical section defines the primary reflective surface 416 and a single conical reflector 414 defines the secondary reflector. The target is positioned behind the apparatus 400 from the perspective of the viewer of the figure and the camera in front of it from that perspective. Spokes 418 may provide support for the secondary reflector 414 and both the primary and secondary reflectors 414 and 416 may be supported in a rigid frame 418 which may be a hollow structure to provide stiffness and precise location of the surfaces. The spokes 418 may be tensioned to hold the secondary reflector rigidly and allow adjustment of the orientation of the secondary reflector 414. The spokes may also be rigid structures that support a chassis adjustment fasteners such as adjustable screws to orient the secondary reflector. The apparatus 400 may be used in a small animal imaging system as shown in FIG. 5A or may be used for the imaging of human anatomy.

Referring to FIG. 5A, a small animal imaging system may include conical first 514 and second 516 reflectors. A motion control apparatus may provide for the relative movement of an illumination source 504 (which may also include a camera for surface geometry acquisition) and a target object 512 such as a small animal. A first motion axis may have a drive 508 that rotates a support arm 506 to position the camera 504 and/or an illumination source 504 . A second drive 511 may position a stage 515 to move the object 512. Other configurations for providing relative movement of the object and/or imaging elements are also possible and the illustration provides merely an example. The conical mirror pair and the CCD camera measure photons from the entire surface of a small animal and by using the motion control part, which is comprised of a rotational gantry and a translational linear stage, any area on the surface of a subject can be illuminated by a pin-point laser source.

Referring to FIG. 5B, an imaging device 531 has flat reflectors 542 and 540 defining primary and secondary reflectors. The configuration of the reflectors defines a bilaterally symmetric arrangement suitable for imaging relatively flat surface regions such as the tarsal region of a foot 536. A structure 534 may support light sources and provide for translation or other movement thereof. The stage may provide an opening or window 532 to allow for the illumination by light source(s) or for diffuse transmitted light to be received.

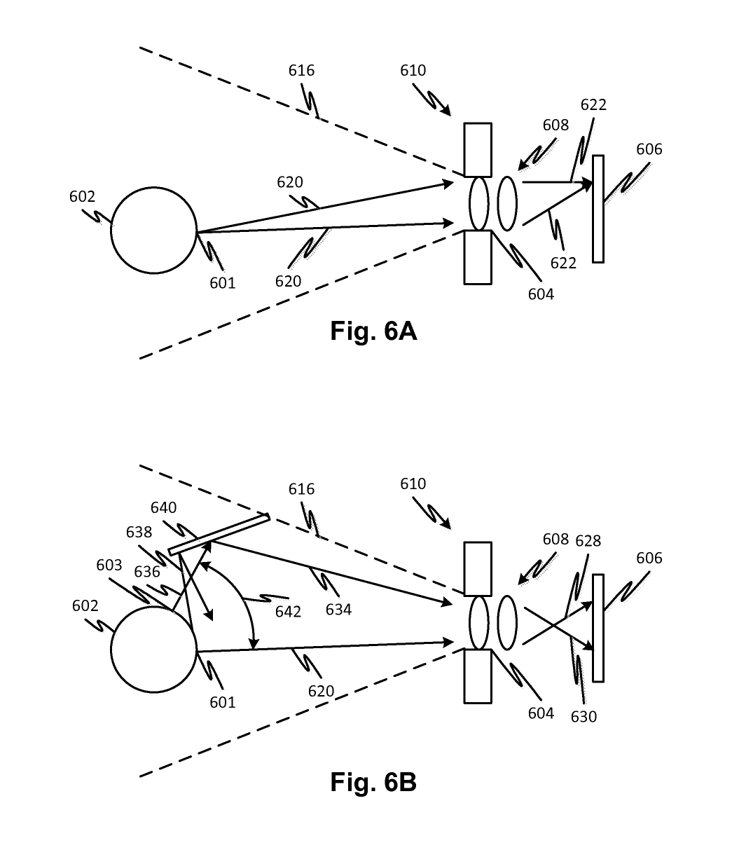

Referring to FIG. 6A, in a normal camera arrangement, diverging light rays 620 from an arbitrary point 601 on an imaged object 602 are reconverged (as indicated by arrow 622) by optics 608 of a camera 610 so that they fall on a common point on a photoreceptive surface 606 such as a CCD. The camera 610 may have a field of view 616 that can take in the object as well as some of the area around the object 602. As shown in FIG. 6B, by placing an optical element such as a minor 640 at a certain orientation and position, a light ray 636 from a point 603 remote from point 601 on the object and diverging from the light ray 620 by a large angle 642 may be directed toward the camera along a path 634 such that the ray is directed 630 to a distinct portion of the photoreceptive surface 606. At the same time, the angle and position of the optical element 640 is such that light from substantially any point on the surface of the object such as ray 638 is still directed away from the object 602.

In the arrangement of FIG. 6B, the angle of divergence of two rays, which would in the absence of the optical element 640, not fall into a capture aperture 604 of the camera 610 is reduced such that both rays 620 and 634 fall within the capture aperture 604 of the camera. This permits sides of the object 602 facing in different directions to be imaged on a single image plane. The embodiment illustrated in FIG. 6B also illustrates that some of the benefits of the prior embodiments may be achieved with a single redirection by an optical element rather than using multiple redirecting elements in series.

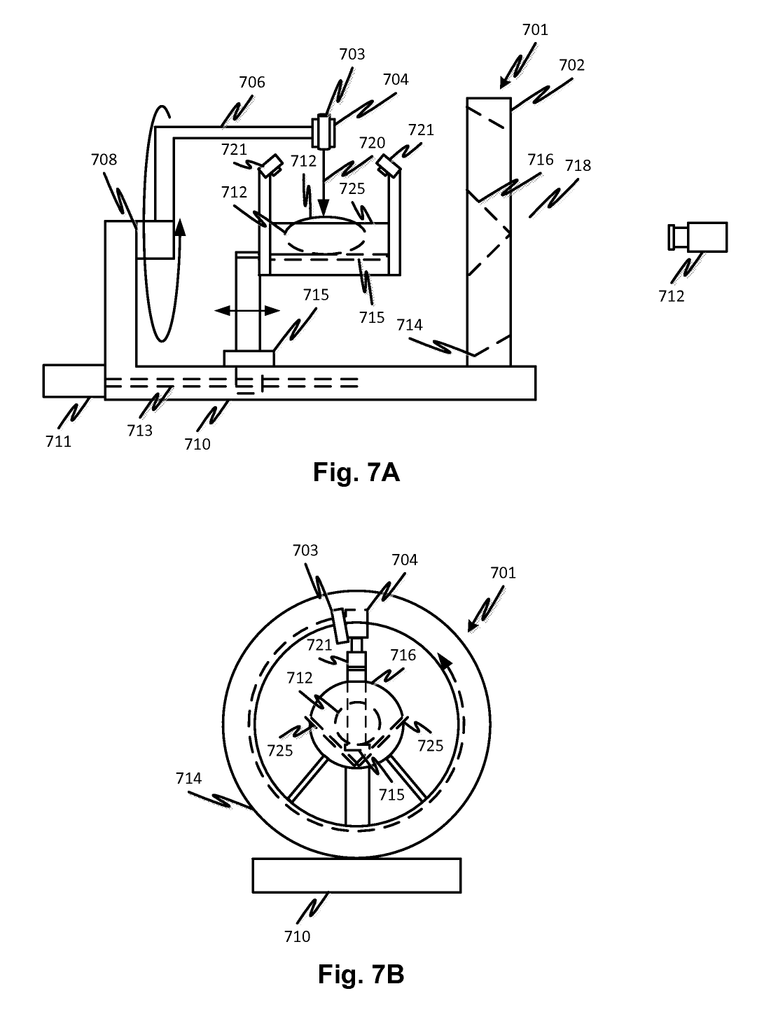

Referring to FIG. 7A, a small animal imaging system may include an imaging component constructed like apparatus 400 in FIG. 4, with conical first 714 and second 716 reflectors. A motion control apparatus may provide for the relative displacement of an illumination source 704 with respect to a target object stage 715. A target object 712 is shown on the stage 715 which may be transparent. The target object 712 may be a small animal. A second illumination source 703 may be a line scanner that scans through an angle perpendicular to the page of the drawing of FIG. 7A and parallel to a plane of the drawing of FIG. 7B. This may be used to produce a scanned line that reveals the shape of the object 712 clearly to cameras 721 aimed thereat.

A pair of mirrors 725 are positioned at angles with respect to each other, for example, 90 to 120 degrees apart as shown best in FIG. 7B. The minors 725 reflect the back and side of the object 712 toward the cameras 721 providing overlapping data that can be processed using known techniques to generate a model of the surface of the object 712 to facilitate the generation of a suitable mesh for tomographic data generation. Drive 729 that rotates a rotational ring 728 to position illumination source 704 in any direction around a target object 712. The second linear drive 727 positions a platform 726 of a pair of minors 725 and two cameras 721 for surface scan. The platform 726 can be traversed by the linear drive 727 after the surface scan. And the light source 704 may be directed to successive rotational positions by the rotational drive 729, after the platform 726 is moved, to map out a selection of source points for tomographic data acquisition. Other configurations for providing relative movement of imaging elements are also possible and the illustration provides merely an example. The conical mirror pair 701 and the CCD camera 712 measure photons from the entire surface of a small animal for each point source generated by a respective position of the first and second drives and the illuminations source 704.

In the described systems, the entire surface of a subject can be observed like a single conical mirror scheme and, because a subject is placed out of a mirror pair structure, the unwanted back reflection effect can be reduced. Moreover, by using empty space around a subject, other modalities such as CT or PET can be combined with this optical imaging system without being disturbed by the minor structures around a subject.

In any of the disclosed embodiments, a light source may be scanned using a DLP type scanner or any other suitable orienting mechanism. In any of the embodiments, target objects may be scanned by providing relative movement of the object and/or light source. In any of the embodiments, the cylindrical and flat optical surfaces may be replaced with non-flat and non-cylindrical surfaces and three-dimensional curves that can produce images. In any of the embodiments, surface geometry may be acquired by laser scanning rather than multiple-vantage surface imaging or by any other means. In any of the disclosed embodiments, a refracting device such as a prism may be used to achieve reflection rather than a mirror. In any of the embodiments, the minors may be include multiple facets rather than a curved surface so as to permit the surrounding, full or partial, of the target object.

In any of the disclosed embodiments, instead of a point illumination and camera imaging being used for tomographic reconstruction, the imaging devices disclosed may also be used for surface acquisition, either using multiple images to create a surface model or by laser scanning, or by any other means.

According to embodiments, the disclosed subject matter includes an imaging system. The system has a target object support that is configured to hold an object. The object itself is not part of the imaging system. The system has a first optical component positioned and configured to receive light from a target positioned on said support and to redirect the received light to a second optical component by aiming the received light in a different direction. The optical component does this by reflection or refraction to direct the light through an air path rather than a light pipe or by conversion to an electronic signal. Effectively this redirecting can allows multiple sides of the object to be imaged by, for example, reflecting light from opposite vantages toward the imaging device (e.g., camera) thereby creating a multiple component redundant or partially redundant view of the object by the camera. The optical component can use multiple reflectors in series in order to allow higher angles of incidence and lower loss of fidelity due to cosine compression of the reflection.

The system can also have a source of illumination configured to direct a light beam from various angles toward said target object support such that an object placed on said object support may be illuminated from multiple sides. The illumination source may be used for surface scanning such as a laser spot or line scan used to create a three-dimensional model of an object as used in high speed inspection systems. The illumination source or a different one may be used to create periodic and variously-located surface sources on the object or deeply seated sources within the object for use in optical tomography. The system may also be used without an illumination source for example if used for tomographically determining a light source (bioluminescent source) distribution within the object.

Ultimately one of the features of the embodiments described above is the fact that the optical component directs the light in a way that avoids reflection of light back onto the object. For example, when a surface source is generated by a light source, for example a laser spot aimed onto the object surface, a good deal of light is reflected from the surface. If any parts of an optical component has a reflector and any part of the reflector can reflect the rays of light from the diffuse reflection from the spot, this will produce more illumination onto the object. This may degrade the optical tomographic signal used for imaging the internal characteristics of the object being interrogated. Even light that is transmitted out of the object can “retro-reflect” from the optical component, such a surface as in bioluminescence tomography. Thus, a first optical element of the optical component may be positioned relative to the target object support such that specular reflection or refraction of light from an object on the target object support, may be directed away from a target object on said target object support, whereby secondary illumination of a target by photons emitted from the surface thereof may be prevented. A second element may facilitate the directing of the reflection into the field of view of the imaging camera.

In embodiments, the first optical element is configured to receive said light from a target from multiple opposing sides of the target. The second element is used to align the image for capture by the camera. The first and second sets of optical elements may include combinations of flat and/or conical minors. For example pyramidal or conical arrangements may be used to partly surround the object.

The system, as an embodiment, may or may not include a camera as part of the system permitting a user to provide his own camera. In embodiments, the camera is included as part of a system. In other embodiments, the system without the camera is used with a camera by the user so it may be delivered without a camera but may include a standard mounting and positioning stage for a camera. The stage may allow the camera to be positioned with respect to the optical component to align its field of view appropriately to achieve the foregoing features.

The imaging system may be supplied with a controller and/or a computer to provide for tomographic data construction. The system may also include a display for the presentation of tomographic constructions for example three-dimensional views of chromophores distributions.

It is, thus, apparent that there is provided, in accordance with the present disclosure, optical methods, devices, and system for optical tomography. Many alternatives, modifications, and variations are enabled by the present disclosure. Features of the disclosed embodiments can be combined, rearranged, omitted, etc., within the scope of the invention to produce additional embodiments. Furthermore, certain features may sometimes be used to advantage without a corresponding use of other features. Accordingly, Applicants intend to embrace all such alternatives, modifications, equivalents, and variations that are within the spirit and scope of the present invention.

1-22. (canceled)

23. An imaging system, comprising:
a target object support;
a first optical component positioned to receive light from a target positioned on said support and to redirect the received light to an imaging device, with a field of view, by aiming the receive light in a different direction;
the first optical component being positioned relative to the target object support such that specular reflection or refraction of light from an object on the target object support, is directed away from a target object on said target object support, whereby secondary illumination of a target by photons emitted from the surface thereof is prevented;
the first optical component being configured to narrow an angle between first and second rays of light aimed away from said target object support and received by said first optical element such that the first and second rays can be directed into said field of view;
a source of illumination configured to direct a light beam from various angles toward said target object support such that an object placed on said object support is illuminated from multiple sides.

24. The imaging system of claim 23, further comprising a second optical element that further redirects said light before it reaches said imaging device.

25. The imaging system of claim 24, wherein the first and second optical components include mirrors.

26. The imaging system of claim 24, wherein the first and second sets of optical elements include combinations of flat and/or conical mirrors.

27. The imaging system of claim 24, wherein the second optical component transfers the image to the imaging device by reflection.

28. The imaging system of claim 23, wherein the imaging device is a CCD or CMOS camera, which reconstructs the target image from the transferred images.

29. The imaging system of claim 23, wherein the first optical components at least partly surrounds an axis that is aligned with the target object support.

30. The imaging system of claim 23, wherein the first optical components fully surrounds an axis that is aligned with the target object support.

31. The imaging system of claim 23, wherein said first optical component includes a conical mirror.

32. The imaging system of claim 31, wherein the conical mirror has an axis and the target object support lies along said axis and displaced beyond a larger end of said conical mirror.

33. An imaging device, comprising:
a target object support;
a first optical component positioned to receive light from a target positioned on said support and to redirect the received light to an imaging device, with a field of view, by aiming the receive light in a different direction;
the first optical component being positioned relative to the target object support such that light, directed in opposite directions, from an object on the target object support that is received thereby, is entirely directed through the ambient air away from a target object on said target object support, whereby secondary illumination of a target by photons emitted from the surface thereof is prevented;
the first optical component being configured to direct said light received thereby to a region defining the field of view of an imaging device.

34. The device of claim 33, further comprising a source of illumination configured to direct a light beam from various angles toward said target object support such that an object placed on said object support is illuminated from multiple sides.

35. The imaging device of claim 33, further comprising a second optical element that further redirects said light before it reaches said imaging device.

36. The imaging device of claim 35, wherein the first and second optical components include mirrors.

37. The imaging device of claim 35, wherein the first and second sets of optical elements include combinations of flat and/or conical mirrors.

38. The imaging device of claim 35, wherein the second optical component transfers the image to the imaging device by reflection.

39. The imaging device of claim 33, wherein the imaging device is a CCD or CMOS camera, which reconstructs the target image from the transferred images.

40. The imaging device of claim 33, wherein the first optical components at least partly surrounds an axis that is aligned with the target object support.

41. The imaging device of claim 33, wherein the first optical components fully surrounds an axis that is aligned with the target object support.

42. The imaging device of claim 33, wherein said first optical component includes a conical mirror.

43. The imaging device of claim 42, wherein the conical mirror has an axis and the target object support lies along said axis and displaced beyond a larger end of said conical mirror.

44. An imaging system comprising
an imaging device, comprising:
a target object support;
a first optical component positioned to receive light from a target positioned on said support and to redirect the received light to an imaging device, with a field of view, by aiming the receive light in a different direction;
the first optical component being positioned relative to the target object support such that light, directed in opposite directions, from an object on the target object support that is received thereby, is entirely directed through the ambient air away from a target object on said target object support, whereby secondary illumination of a target by photons emitted from the surface thereof is prevented;
the first optical component being configured to direct said light received thereby to a region defining the field of view of an imaging device; and
an imaging device and a processor programmed to generate optical tomographic data from images received by said imaging device.

45-51. (canceled)
